(19) United States
(12) Patent Application Publication
(10) Pub. No.: US 2018/0036494 A1
(43) Pub. Date: Feb. 8, 2018

(54) THERMAL TUBING

(71) Applicant: Abraham Taub, Spring Valley, NY (US)

(72) Inventor: Abraham Taub, Spring Valley, NY (US)

(21) Appl. No.: 15/551,498

(22) PCT Filed: Feb. 5, 2016
(86) PCT No.: PCT/US16/16714

§ 371 Date: Aug. 16, 2017

Related U.S. Application Data
(60) Provisional application No. 62/118,708, filed on Feb. 20, 2015.

(51) Int. Cl. A61M 5/44; A47J 31/00; A61L 29/14; A61M 39/08; A61M 5/14

(52) U.S. Cl. CPC A61M 5/44; A61M 39/08; A61M 5/1411; A61L 29/14; A47J 31/00; A61M 2205/364; A61M 2205/3633; A61M 2205/3606

(57) ABSTRACT

A device includes a thermal element capable of instantly activating to change a temperature of the thermal element for thermal transfer between the thermal element and a fluid, and tubing made of a flexible material having a hollow core that forms a conduit for a flow of the fluid through the tubing. At least a portion of the tubing may be coiled around the thermal element to provide a predetermined surface area of engagement between the tubing and the thermal element for thermal transfer between the thermal element and the fluid when the thermal element is instantly activated and the fluid is flowing through the tubing.

### CROSS-REFERENCE TO RELATED APPLICATIONS

This application is a national stage entry application of International Application No. PCT/US16/16714 filed on Feb. 5, 2016, which claims priority to U.S. Provisional App. No. 62/118,708 filed on Feb. 20, 2015, where the entire contents of each of the foregoing is hereby incorporated by reference.

### TECHNICAL FIELD

The present disclosure generally relates to thermal tubing, and more specifically to thermal tubing for an intravenous (IV) drip system.

### BACKGROUND

In the medical field, there are many conditions that call for the intravenous administration of warmed or cooled fluids and blood products, e.g., cooled fluids for therapeutic hypothermia in post-cardiac arrest or warmed fluids for hypothermic and trauma patients. In fact, the current standard of care in the operating room is to administer warm fluids for patients during surgery. As such, there are electrical pumps and devices utilized in hospitals to cool or warm fluids and blood products for administering to patients. However, in the prehospital setting (e.g., during transport to the hospital), many of these devices are impractical due to their size or power-supply requirements. By way of example, in a trial study of therapeutic hypothermia in the New York City Emergency Medical Services (EMS) system, small refrigerators were installed in participating ambulances to cool fluids and maintain a desired temperature, but this was found to be a generally expensive and cumbersome endeavor. Additionally, while the current standard of care calls for the administration of warm fluids to hypothermic patients, EMS providers in the field, during the winter months in cold weather climates, are instructed to keep a bag of saline on the ambulance's dashboard to be heated by the ambulance's vents and the sun because affordable, convenient methods for thermal control of fluids in the field do not exist in the prior art. There thus remains a need for practical and portable thermal control of intravenous fluids in the field for emergency response personnel and the like. There also remains a need for practical and portable thermal control of fluids in other industries, e.g., the beverage industry.

### SUMMARY

A device includes a thermal element capable of instantly activating to change a temperature of the thermal element for thermal transfer between the thermal element and a fluid, and tubing made of a flexible material having a hollow core that forms a conduit for a flow of the fluid through the tubing. At least a portion of the tubing may be coiled around the thermal element to provide a predetermined surface area of engagement between the tubing and the thermal element for thermal transfer between the thermal element and the fluid when the thermal element is instantly activated and the fluid is flowing through the tubing.

Implementations may include one or more of the following features. The device of may include an insulating wrapper disposed around the thermal element and the portion of the tubing coiled around the thermal element. The tubing may include a first end and a second end, where the first end and the second end each protrude from the insulating wrapper. The first end may include a first connector and the second end may include a second connector, where each of the first and second connectors is configured for engagement with components of a fluid flow system. The fluid flow system may be an intravenous (IV) drip system. The tubing may include intravenous (IV) tubing. The predetermined surface area of engagement between the tubing and the thermal element may be selected to heat or cool fluid exiting the tubing to a predetermined temperature. The thermal element may include at least one of a hot pack or a cold pack. The thermal element may be activated without the use of an external power source. The thermal element may include contents capable of instantly activating through agitation of the contents to change the temperature of the thermal element. The thermal element may be battery powered. A temperature of the thermal element may be controllable. The fluid may be selected from the group consisting of a sodium chloride solution, a dextrose solution, oxygen, and a blood product. The tubing may include at least one of polypropylene, nylon, and dynaflex. The fluid may include a consumable fluid of a beverage. The predetermined surface area of engagement may be adjustable by increasing or decreasing an amount of coiling of the tubing around the thermal element.

In one aspect, an intravenous (IV) drip system includes an IV bag containing a fluid, a thermal element capable of instantly activating to change a temperature of the thermal element for thermal transfer between the thermal element and the fluid between the IV bag and a patient, and IV tubing having a hollow core that forms a conduit for a flow of the fluid through the IV tubing from the IV bag to the patient. At least a portion of the IV tubing may be coiled around the thermal element to provide a predetermined surface area of engagement between the IV tubing and the thermal element for thermal transfer between the thermal element and the fluid received by the patient when the thermal element is instantly activated and the fluid is flowing through the IV tubing.

Implementations may include one or more of the following features. The system may further include an insulating wrapper disposed around the thermal element and the portion of the IV tubing coiled around the thermal element. The thermal element may include contents capable of instantly activating through agitation of the contents to change the temperature of the thermal element.

In another aspect, a method includes coiling at least a portion of tubing around a thermal element, the tubing having a hollow core that forms a conduit for a flow of fluid through the tubing, the thermal element capable of instantly activating to change a temperature of the thermal element for thermal transfer between the thermal element and fluid in the tubing; wrapping an insulating wrapper around the portion of the tubing coiled around the thermal element such that a first end of the tubing and a second end of the tubing are unwrapped and exposed to facilitate a connection of the tubing with a fluid flow system; and providing a first connector on the first end of the tubing and a second connector on the second end of the tubing, the first and second connectors configured for engagement with components of the fluid flow system.

In yet another aspect, a method includes coiling at least a portion of intravenous (IV) tubing around a thermal element, the IV tubing having a hollow core that forms a conduit for a flow of fluid through the IV tubing, the thermal element capable of instantly activating to change a temperature of the thermal element for thermal transfer between the thermal element and the fluid in the IV tubing; connecting the IV tubing to an IV drip system; activating the thermal element; and providing the flow of fluid using the IV drip system.

In another aspect, a device includes tubing made of a flexible material and having a hollow core that forms a conduit for a flow of fluid through the tubing, and a thermal element disposed around the tubing to provide a predetermined surface area of engagement between the tubing and the thermal element. The thermal element may be capable of instantly activating to change a temperature of the thermal element for thermal transfer between the thermal element and fluid flowing through the tubing.

Implementations may include one or more of the following features. The tubing may include a first end having a first connector and a second end having a second connector, where each of the first and second connectors is configured for engagement with components of a fluid flow system. The fluid flow system may be an intravenous (IV) drip system or a beverage consumption system.

### BRIEF DESCRIPTION OF THE DRAWINGS

The foregoing and other objects, features and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein.

FIG. 1 depicts an intravenous (IV) drip system.

FIG. 2 is a device featuring a thermal element engaged with tubing.

FIG. 3 is a side view of a thermal element engaged with tubing.

FIG. 4 depicts a thermal tubing system.

FIG. 5 is a top view of thermal tubing.

FIG. 6 is a side view of thermal tubing.

FIG. 7 is a flow chart of a method for making thermal tubing.

FIG. 8 is a flow chart of a method for using thermal tubing in an IV drip system.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the context. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the embodiments.

In the following description, it is understood that terms such as “first,” “second,” “top,” “bottom,” “up,” “down,” and the like, are words of convenience and are not to be construed as limiting terms unless specifically stated to the contrary.

Described herein are devices, systems, kits, and methods for practical and portable thermal control, e.g., thermal control of intravenous fluids in the field for emergency response personnel and thermal control of fluids in other industries (e.g., the beverage industry). In general, a portable device may include tubing (or a container) engaged with an instantaneous activated hot-pack or cold-pack that is used to heat or cool fluids in the tubing (or container).

The devices, systems, kits, and methods may offer Emergency Medical Services (EMS) providers and technicians in the field with a fairly easy and portable method to administer warmed or cooled fluids without being restricted to large power operated refrigerators, warmers, and pumps. In addition to its advantages in the medical field, the devices, systems, kits, and methods can also or instead be used to benefit the general public in many different applications, such as by creating a portable system for heating and cooling beverages. One skilled in the art will understand that the embodiments described herein may be adapted and configured for use across many industries and applications, all of which are intended to fall within the scope of this disclosure.

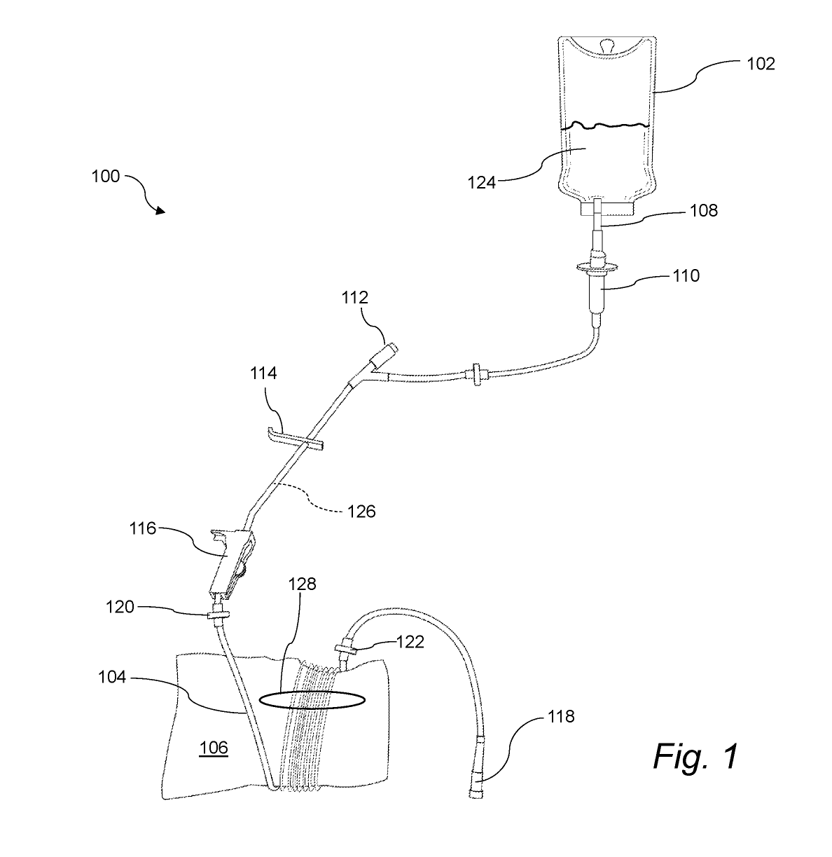

FIG. 1 depicts an intravenous (IV) drip system. The IV drip system 100 may include an IV bag 102, IV tubing 104, and a thermal element 106. The IV drip system 100 may also or instead include other components commonly found in such a system, including without limitation, a bag spike 108 for an insertion point to a fluid source such as the IV bag 102, a drip chamber 110, an injection port 112 (e.g., to piggy-back another IV line or administer medications), auxiliary clamps 114, roller clamps 116 (e.g., for controlling drip rate), an IV catheter connecting point 118, other connecting points (e.g., a first connector 120 and a second connector 122), and so forth.

The IV bag 102 may be the source of fluid 124 for the IV drip system 100. The IV bag 102 may be any as known in the art, or any suitable container for holding the fluid 124 for use in the IV drip system 100.

The fluid 124 may include any fluid or liquid adapted for use in an IV system, including without limitation, a sodium chloride solution, a dextrose solution, a blood product, and so forth. The fluid 124 may also or instead include oxygen or other gases in systems that are adapted for such uses. For example, an embodiment includes thermal tubing for administering oxygen through a breathing tube, face mask, nasal cannula, and so forth.

The IV tubing 104 may have a hollow core 126 that forms a conduit for a flow of the fluid 124 through the IV tubing 104, e.g., from the IV bag 102 to a patient. The IV tubing 104 may be made from any suitable material, including without limitation, one or more of polypropylene, nylon, dynaflex, and the like.

The thermal element 106 may be capable of instantly activating to change a temperature of the thermal element 106, e.g., for thermal transfer between the thermal element 106 and the flow of fluid 124 in the IV tubing 104. In this manner, activating the thermal element 106 may either warm or cool the flow of fluid 124 in the IV tubing 104, e.g., between the IV bag 102 and a patient. For example, the thermal element 106 may include a standard hot pack or cold pack as known in the art. These may include, for example, instant hot or cold packs, chemical heat packs (e.g., sodium-acetate), chemical cold packs (e.g., ammonium nitrate, calcium ammonium nitrate or urea), reusable hot or cold packs (i.e., multi-use), disposable hot or cold packs (e.g., single-use), and so forth. The thermal element 106 may be activated upon agitation of its contents. The thermal element 106 may be shaped and sized for use in the desired system, e.g., the IV drip system 100. For example, the shape of the thermal element 106 may include a tube, a cylinder, a box, a strip, and so forth. The thermal element 106 may also or instead be shaped like a package, sack, or bag, where the package, sack, or bag includes chemical contents for activation through agitation of the chemical contents by inciting an exothermic or endothermic chemical reaction.

In the IV drip system 100, the thermal element 106 may be engaged with the IV tubing 104.

In one aspect, at least a portion of the IV tubing 104 is coiled around the thermal element 106 (forming a number of coils 128) to provide a predetermined surface area of engagement between the IV tubing 104 and the thermal element 106 for thermal transfer between the thermal element 106 and the fluid 124 received by a patient when the thermal element 106 is instantly activated and the fluid 124 is flowing through the IV tubing 104 (i.e., warming or cooling the fluid 124). The predetermined surface area of engagement between the IV tubing 104 and the thermal element 106 may be selected to heat or cool fluid 124 exiting the IV tubing 104 to a predetermined temperature.

In an aspect, the predetermined surface area of engagement is adjustable. For example, the predetermined surface area of engagement may be adjusted by increasing or decreasing an amount of coiling of the IV tubing 104 around the thermal element 106 (i.e., the number of coils 128). In other words, a predetermined amount of coiling around the thermal element 106 may provide a predetermined surface area of engagement selected for specific heating or cooling of the fluid 124 for a specific application. Adjusting an amount of coiling of the IV tubing 104 around the thermal element 106 may be done manually by an end user (e.g., a medical technician in the field), or by a manufacturer of the devices or systems as described herein.

An implementation may provide guidance for the amount of coiling around the thermal element 106 to be used for specific applications. The guidance may be in the form of a computer program product including computer executable code embodied in a non-transitory computer readable medium that, when executing on one or more computing devices, calculates and provides such guidance based on certain variables. The variables may include without limitation the fluid 124 to be delivered, the type and size of the tubing, the type and size of the thermal element 106, the circumstances for delivery (e.g., a hypothermic patient, a surgical application, and so forth), information regarding a patient (e.g., age, weight, etc.), an external temperature, and so forth. In an aspect, these variables may be inputted into a computing device that then uses processing circuitry to determine an amount of coiling to be used and displays the same for use by an end user or the like. Determination of the amount of coiling to be used may be computed using thermodynamic equations and calculations known in the art.

In an aspect, the predetermined temperature is also or instead adjustable. The predetermined temperature may be adjusted through an adjustment of the amount of coiling around the thermal element 106 as described above. The predetermined temperature may also or instead be adjusted by control or manipulation of the thermal element 106. For example, a predetermined amount of agitation may be used, a time period for using the thermal element 106 may be adjusted, or the thermal element 106 may be controlled by a controller or the like.

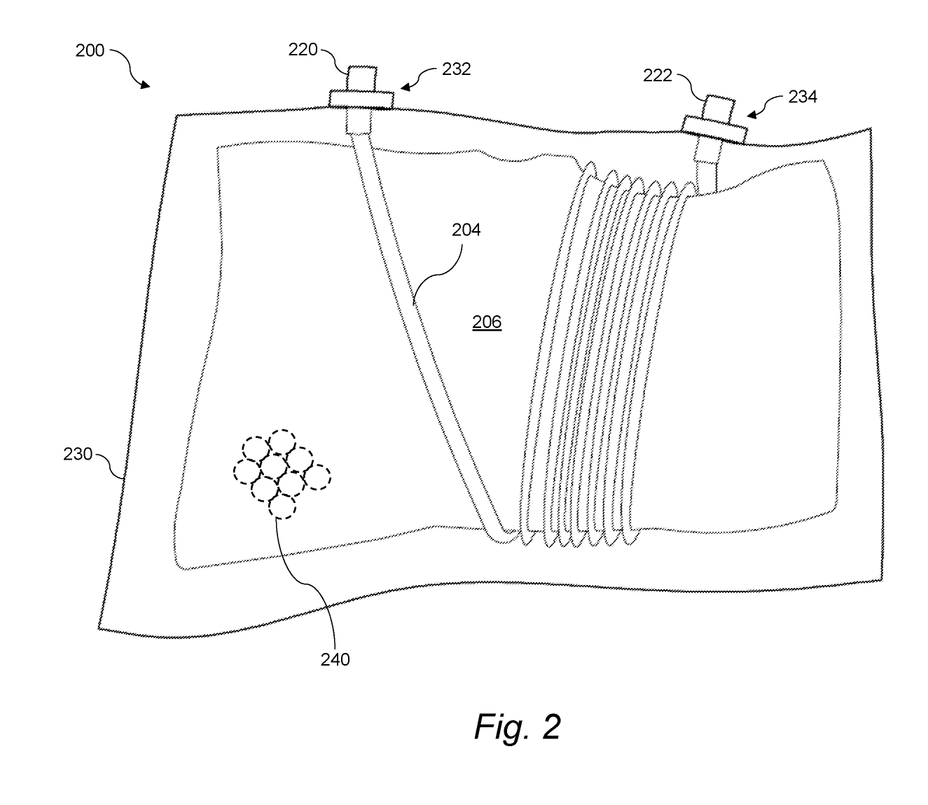

FIG. 2 is a device featuring a thermal element engaged with tubing. As shown in the figure, the device 200 may include tubing 204 coiled around a thermal element 206.

The device 200 may be a portable device that is attachable to an IV drip system (such as the system described above) or other fluid delivery system. The device 200 may thus be shaped and sized such that it is portable and adaptable to connect as an extension to a fluid delivery system, e.g., a standard IV drip system. Because of the inclusion of the thermal element 206, the device may instantly activate, e.g., when the contents of the thermal element 206 are agitated, in order to cool or warm fluids that flow through the tubing 204 of the device 200 and the fluid delivery system.

The tubing 204 may be made of a flexible material. The tubing 204 may include a hollow core that forms a conduit for a flow of a fluid through the tubing 204. As shown in the figure, at least a portion of the tubing 204 may be coiled around the thermal element 206 to provide a predetermined surface area of engagement between the tubing 204 and the thermal element 206 for either warming or cooling the fluid when the thermal element 206 is activated and the fluid is flowing through the tubing 204. The predetermined surface area of engagement between the tubing 204 and the thermal element 206 may be selected to heat or cool fluid exiting the tubing 204 to a predetermined temperature, e.g., for delivery to a patient in an IV drip system such as that described above. The predetermined surface area of engagement may be adjustable by increasing or decreasing an amount of coiling of the tubing 204 around the thermal element 206.

The tubing 204 may include IV tubing or the like. The tubing 204 may be made of one or more of a polypropylene, a nylon, a dynaflex, and the like. The fluid flowing through the tubing 204 may include a sodium chloride solution, a dextrose solution, oxygen, a blood product, and the like. In another aspect, the fluid includes a consumable fluid of a beverage.

The thermal element 206 may be capable of instantly activating to change a temperature of the thermal element 206 for thermal transfer between the thermal element 206 and a fluid. In this manner, when activated, the thermal element 206 may either warm or cool a fluid, e.g., a fluid flowing through the tubing 204. The thermal element 206 may include at least one of a hot pack or a cold pack. The thermal element 206 may be activated without the use of an external power source. For example, the thermal element 206 may include contents 240 capable of instantly activating through agitation of the contents 240 to change the temperature of the thermal element 206 for thermal transfer, e.g., either warming or cooling the fluid flowing through the tubing 204. The thermal element 206 may also or instead be battery powered, solar powered, or the like. The thermal element 206 may also or instead be chargeable.

The device may include a wrapper 230. The wrapper 230 may include an insulating wrapper disposed around the thermal element 206 and the portion of the tubing 204 coiled around the thermal element 206. In this manner, the device 200 may be covered, shrouded, wrapped, or otherwise engaged with an insulating material, e.g., the wrapper 230. The wrapper 230 may serve to insulate the device 200, protect the device 200, and/or create a neat, small portable handheld device 200 for transport. The wrapper 230 may also or instead wrap the device 200 and hold the tubing 204 in place where it is engaged with the thermal element 206.

Ends of the tubing 204 (e.g., a first end 232 and a second end 234) may protrude from the wrapper 230. To facilitate connection to a fluid delivery system, the ends of the tubing 204 may include connectors (i.e., a first connector 220 and a second connector 222 as shown in the figure). Specifically, the tubing 204 may include a first end 232 including a first connector 220 and a second end 234 including a second connector 222, where the first end 232 and the second end 234 each protruding from the wrapper 230. The first connector 220 and the second connector 222 may be configured for engagement with components of a fluid flow system. For example, the connectors may include standard IV connection devices, e.g., injection ports and connecting points. Thus, both sides of the device 200 may include standard IV connection ports, such that the device 200 can be placed between administered fluids and a patient.

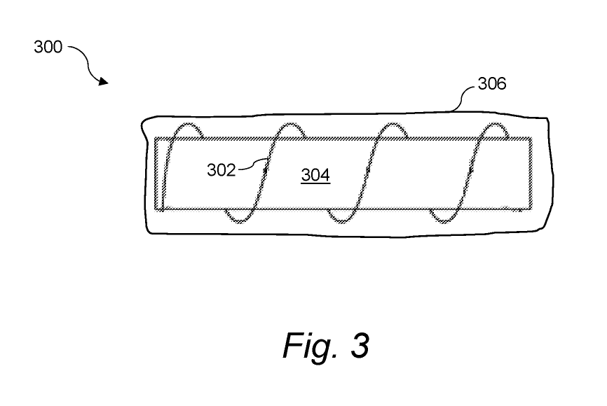

FIG. 3 is a side view of a thermal element engaged with tubing. In general, the device 300 shown may include a first element 302 coiled around a second element 304. As described in more detail below, in an aspect, the first element 302 may include tubing and the second element 304 may include a thermal element. In another aspect, the first element 302 may include a thermal element and the second element 304 may include tubing. Thus, in general, FIG. 3 is a representation of a device 300 including tubing and a thermal element, where either the tubing is coiled around the thermal element or vice-versa.

The tubing may include a hollow core that forms a cavity for a flow of fluid through the tubing. In general, the tubing may be any such that fluid may flow from a first point to a second point through the tubing. The first point may include a fluid source, and the second point may include an endpoint. In the example of the IV system as contemplated herein, the fluid source may include an IV bag or the like. The fluid source may also or instead include a gas tank (e.g., an oxygen tank), a canteen, a flexible or rigid fluid container, and so forth. The endpoint may include any desirable destination for the fluid, including without limitation, a user, a patient, a container, and so forth.

The tubing may include a flexible plastic material, including without limitation, IV tubing made of at least one of polypropylene, nylon, and dynaflex. The tubing may also or instead include other materials such as metal, ceramic, paper, a fibrous material, and so forth. The tubing may also or instead include a rigid material. In an aspect, the tubing is resistant to melting.

The thermal element may be engaged with the tubing and capable of instantly activating to change a temperature of the thermal element for thermal transfer between the thermal element and a flow of fluid in the tubing. The thermal element may include a hot pack or a cold pack. The thermal element may be shaped and sized for its specific purpose and for its specific configuration with the tubing of the device. The thermal element may be selected to heat or cool fluid in the tubing to a predetermined temperature, e.g., a temperature desirable for IV fluids or consumption.

The thermal element may be activated independent from an external power source. For example, the thermal element may be manually activated through agitation of the contents included in the thermal element. This may occur through, e.g., shaking, squeezing, torqueing, moving, applying another force or pressure, and so forth. Thus, the thermal element may be activated through manual power by a user. The thermal element may also be engaged with a mechanical means that provides for agitation of the contents of the thermal element or otherwise activates the thermal element, e.g., a spring loaded element that applies a force to the thermal element upon activation by a user.

The thermal element may also or instead be battery powered, or powered by other power sources (e.g., solar, electric, wind, chemical, and so forth).

The thermal element may be controllable, e.g., to provide a desired temperature or temperature range.

The tubing may wrap around the thermal element such that the tubing forms a coil along a longitudinal axis of the thermal element. In this manner, in FIG. 3, the first element 302 may include the tubing and the second element 304 may include the thermal element, where the tubing coils around the thermal element. The thermal element may also or instead be engaged to an exterior of the tubing along a length of the tubing, where the length of the tubing is selected to heat or cool fluid exiting the length of the tubing to a predetermined temperature. In this manner, in FIG. 3, the first element 302 may include the thermal element and the second element 304 may include the tubing, where the thermal element coils around the tubing.

The device 300 may further include insulation in the form of a wrapper 306, which may be included on one or both of the tubing and thermal element. The wrapper 306 may insulate the device 300, protect the device 300, enable engagement of the tubing and thermal element, and so forth.

The device 300 may be part of a kit, including without limitation, a kit for an IV system, a kit for a thermal drinking system, and so forth. The device 300 may thus include features to enable connection of the device 300 to other components of the kit or system. In one aspect, the device 300 includes one or more connectors configured to engage either or both of the tubing and the thermal element to other components of the kit or system, e.g., a standard IV drip system or a beverage container.

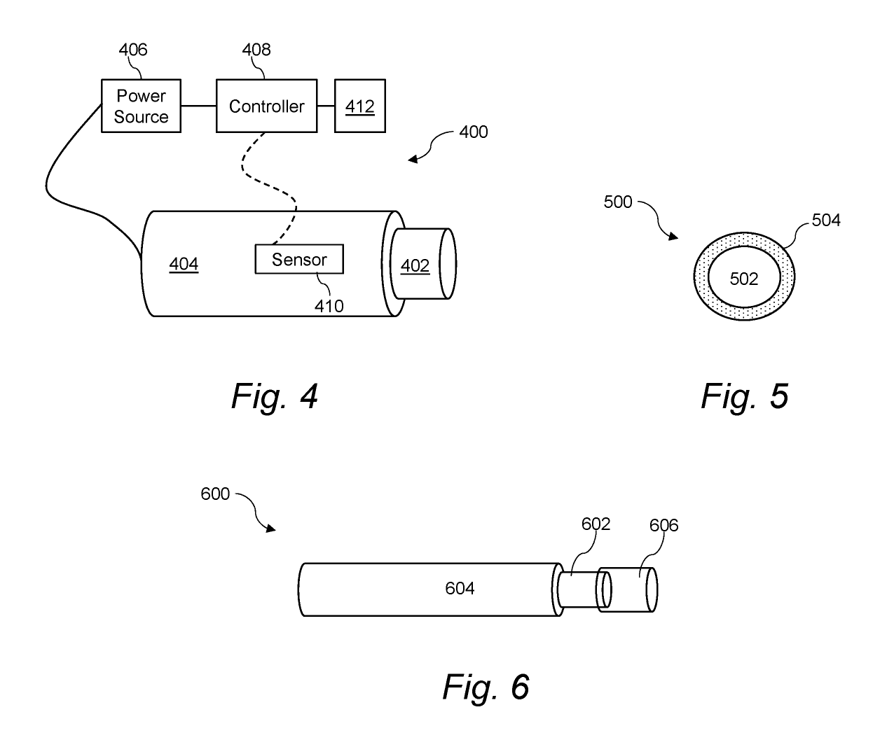

FIG. 4 depicts a thermal tubing system. The thermal tubing system 400 may include tubing 402, a thermal element 404, a power source 406, a controller 408, and a sensor 410.

The power source 406 may include any as described herein, e.g., a battery.

The controller 408 may control a temperature of the system 400. For example, the controller 408 may connect to or send a signal to either or both of the power source 406 and the thermal element 404, e.g., to control a temperature of the thermal element 404. The controller 408 may also or instead send control signals to other components of the system 400. The controller 408 may be configured to receive feedback from components of the system 400 (e.g., sensors 410 such as temperature sensors), and to receive instructions from a user. The controller 408 may be electrically or otherwise coupled in a communicating relationship with one or more components of the overall system 400. The controller 408 may include any combination of software and/or processing circuitry suitable for controlling the various components of the system 400 described herein including without limitation microprocessors, microcontrollers, application-specific integrated circuits, programmable gate arrays, and any other digital and/or analog components, as well as combinations of the foregoing, along with inputs and outputs for transceiving control signals, power signals, sensor signals, and so forth. In one aspect, this may include circuitry directly and physically associated with the components of the system 400, such as a processor or memory (generally depicted as element 412 in the figure). In another aspect, this may be a processor, which may be associated with a personal computer or other computing device coupled to the components of the system, e.g., through a wired or wireless connection. Similarly, various functions described herein may be allocated between a controller, processor, and a separate computer. All such computing devices and environments are intended to fall within the meaning of the term “controller” or “processor” as used herein, unless a different meaning is explicitly provided or otherwise clear from the context.

As shown in the figure, the thermal element 404 may wrap around the tubing 402 forming a sleeve or the like. The sleeve shown in the figure may also or instead include insulation that wraps around the tubing or thermal element. Although the figure generally depicts the thermal element 404 wrapped around the tubing 402, other configurations are possible such as those described elsewhere herein (e.g., where the tubing coils around the thermal element). Thus, one or more of the components of the system 400 such as the power source 406, the controller 408, and the sensor 410 may be used with other configurations as described herein.

FIG. 5 is a top view of thermal tubing. In particular, FIG. 5 shows a device 500 having a first element 502 and a second element 504 surrounding the first element 502. In one aspect, the first element 502 is the tubing as contemplated herein and the second element 504 is the thermal element that forms a sleeve around all or a portion of the exterior of the tubing 502. In another aspect, the first element 502 is either or both of the thermal element and the tubing, and the second element 504 is insulation.

In yet another aspect, the first element 502 is a container including a cavity for holding a beverage, and the second element 504 is a thermal element engaged with the container, where the thermal element is capable of instantly activating to either warm or cool the beverage included in the cavity of the container. In this embodiment, the fluid as contemplated herein may include a consumable fluid of a beverage, including without limitation, water, beer, juice, tea, coffee, soda, soup, and so forth. The container may include a cup, bottle, can, or the like.

FIG. 6 is a side view of thermal tubing. Specifically, FIG. 6 shows a device 600 including tubing 602, a thermal element 604 surrounding the tubing 602, and a connector 606.

The tubing 602 may be made of a flexible material and have a hollow core that forms a conduit for a flow of fluid through the tubing 602.

The thermal element 604 may be disposed around the tubing 602 to provide a predetermined surface area of engagement between the tubing 602 and the thermal element 604. The thermal element 604 may be capable of instantly activating to either warm or cool fluid flowing through the tubing 602.

The tubing 602 may include at least one connector 606. The connector 606 may be any known in the art for connecting the tubing 602 to other tubing, a fluid source, an endpoint, a container, and so forth. In one aspect, the tubing 602 includes a first end having a first connector and a second end having a second connector. Each of the first and second connectors may be configured for engagement with components of a fluid flow system, e.g., an IV drip system or a beverage consumption system.

The devices, systems, and kits described herein may be reusable or they may be made for single-use applications, e.g., disposable.

The devices, systems, and kits described herein may be suitable for use with oxygen tubing or the like, e.g., to administer warm air in a prehospital environment to achieve the standard of care that is currently afforded in the intra-hospital setting through complicated ventilators that administer warm oxygen.

The devices, systems, and kits described herein may provide distinct advantages over simply heating or cooling a fluid source that is then provided through tubing (e.g., heating or cooling an IV drip bag, but not the tubing). This is because, using an implementation as described herein, more fluid may come into thermal contact with the thermal element, e.g., in an aspect where tubing is coiled around the thermal element. This can reduce the time to heat or cool fluid in such systems, and provide an overall more effective and convenient technique.

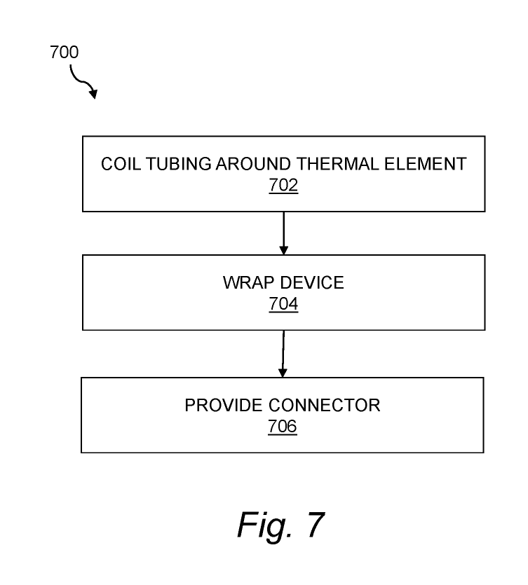

FIG. 7 is a flow chart of a method for making thermal tubing. The method 700 may be used to make a device that is used in conjunction with any IV drip systems as contemplated herein.

As shown in step 702, the method 700 may include coiling at least a portion of tubing around a thermal element. The tubing may have a hollow core that forms a conduit for a flow of fluid through the tubing. The thermal element may be capable of instantly activating to either warm or cool the fluid in the tubing.

The tubing and thermal element may also or instead be otherwise engaged as described herein. For example, engaging the tubing with the thermal element may include wrapping the thermal element around the tubing.

As shown in step 704, the method 700 may include wrapping an insulating wrapper around the portion of the tubing coiled around the thermal element such that a first end of the tubing and a second end of the tubing are unwrapped and exposed to facilitate a connection of the tubing with a fluid flow system.

As shown in step 706, the method 700 may include providing a first connector on the first end of the tubing and a second connector on the second end of the tubing. The first and second connectors may be configured for engagement with components of the fluid flow system. The first and second connectors may be the same connectors or different connectors. The connectors may be configured to engage the tubing to a source of fluid and/or to a patient/user.

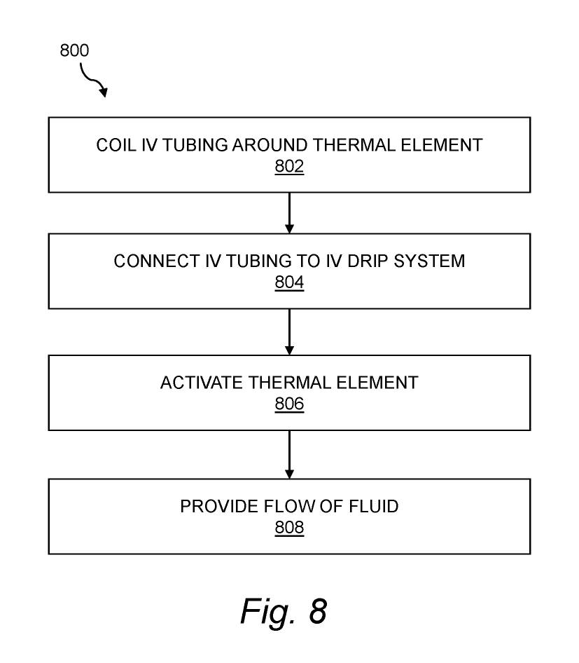

FIG. 8 is a flow chart of a method for using thermal tubing in an IV drip system.

As shown in step 802, the method 800 may include coiling at least a portion of IV tubing around a thermal element. The IV tubing may have a hollow core that forms a conduit for a flow of fluid through the IV tubing. The thermal element may be capable of instantly activating to either warm or cool the fluid in the IV tubing.

As shown in step 804, the method 800 may include connecting the IV tubing to an IV drip system.

As shown in step 806, the method 800 may include activating the thermal element. This may be done by agitating contents included therein.

As shown in step 808, the method 800 may include providing a flow of fluid using the IV drip system.

In the above systems, devices, kits, and methods, the particular type of thermal element (e.g., hot pack or cold pack) may be determined dependent upon one or more of use, size, portability, cost, and other factors. Additionally, the engagement between the tubing and the thermal element may depend upon any or all of the aforementioned factors. For example, in an embodiment where the tubing is wrapped around the thermal element or vice-versa, the number of times a component is wrapped around another component may depend on the ideal surface area of contact between the components to achieve desired temperatures. Other factors may also or instead be considered such as the length of the tubing.

The above systems, devices, kits, and methods may be further adapted for use in the beverage industry. In the beverage industry, drinks are rarely consumed without regard for temperature. Whether coffee, beer, or another beverage, consumers are very particular about the temperature of their drinks and frequently utilize standard household appliances such as percolators and refrigerators to achieve desired temperatures. Similarly, when on-the-go with no easy access to supply for power-consuming appliances, consumers typically cool or heat their drinks in advance, and make use of devices such as a cooler, a thermos, and a travel mug to insulate their beverages and maintain temperatures. Instead, the systems, devices, kits, and methods herein may be adapted for actively heating or cooling liquid without the use of electricity and more efficiently than these standard methods.

For example, a user that goes camping with no access to a power supply may utilize a modified version of the devices described herein instead of carrying a cooler to maintain the cold temperature of liquid beverages. The above device may be modified so that the desired liquid is poured into a container that connects to tubing and runs through a thermal element to achieve a drink at a desired temperature at the receiving end. For example, the thermal tubing may be adapted for consumption of a beverage, where the thermal tubing includes a straw, a conduit for pouring a beverage, and the like. In addition, this concept can be implemented in a manner such that a thermal element is coiled or otherwise disposed inside a container to heat/cool and maintain a temperature of a beverage. As such, the embodiments described herein can allow for cooling liquids without having to dilute them with ice or carrying them in large/heavy ice bags/boxes, or can allow for warm liquids without the need for a power supply or carrying a thermos that only maintains temperature for a finite period of time.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. At the same time, processing may be distributed across devices such as the various systems described above, or all of the functionality may be integrated into a dedicated, standalone device. All such permutations and combinations are intended to fall within the scope of the present disclosure.

Embodiments disclosed herein may include computer program products comprising computer-executable code or computer-usable code that, when executing on one or more computing devices, performs any and/or all of the steps of the control systems described above. The code may be stored in a non-transitory fashion in a computer memory, which may be a memory from which the program executes (such as random access memory associated with a processor), or a storage device such as a disk drive, flash memory or any other optical, electromagnetic, magnetic, infrared or other device or combination of devices. In another aspect, any of the control systems described above may be embodied in any suitable transmission or propagation medium carrying computer-executable code and/or any inputs or outputs from same.

It will be appreciated that the devices, systems, and methods described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to include any suitable method of causing such method steps to be performed, consistent with the patentability of the following claims, unless a different meaning is expressly provided or otherwise clear from the context. So for example performing the step of X includes any suitable method for causing another party such as a remote user, a remote processing resource (e.g., a server or cloud computer) or a machine to perform the step of X. Similarly, performing steps X, Y and Z may include any method of directing or controlling any combination of such other individuals or resources to perform steps X, Y and Z to obtain the benefit of such steps. Thus method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

1. A device comprising:
a thermal element including chemical contents capable of instantly activating through agitation of the chemical contents inciting a chemical reaction to change a temperature of the thermal element for thermal transfer between the thermal element and a fluid; and
tubing made of a flexible material and having a hollow core that forms a conduit for a flow of the fluid through the tubing, wherein at least a portion of the tubing is coiled around the thermal element to provide a predetermined surface area of engagement between the tubing and the thermal element for thermal transfer between the thermal element and the fluid when the thermal element is instantly activated and the fluid is flowing through the tubing.

2. The device of claim 1 further comprising an insulating wrapper disposed around the thermal element and the portion of the tubing coiled around the thermal element.

3. The device of claim 2 wherein the tubing includes a first end and a second end, the first end and the second end each protruding from the insulating wrapper.

4. The device of claim 3 wherein the first end includes a first connector and the second end includes a second connector, and wherein each of the first and second connectors is configured for engagement with components of a fluid flow system.

5. The device of claim 4 wherein the fluid flow system is an intravenous (IV) drip system.

6. The device of claim 1 wherein the tubing includes intravenous (IV) tubing.

7. The device of claim 1 wherein the predetermined surface area of engagement between the tubing and the thermal element is selected to heat or cool fluid exiting the tubing to a predetermined temperature.

8. The device of claim 1 wherein the thermal element includes at least one of a hot pack or a cold pack.

9. The device of claim 1 wherein the thermal element is activated without use of an external power source.

10-11. (canceled)

12. The device of claim 1 wherein the temperature of the thermal element is controllable.

13. (canceled)

14. The device of claim 1 wherein the tubing includes at least one of polypropylene, nylon, and dynaflex.

15. (canceled)

16. The device of claim 1 wherein the predetermined surface area of engagement is adjustable by increasing or decreasing an amount of coiling of the tubing around the thermal element.

17. An intravenous (IV) drip system comprising:
an IV bag containing a fluid;
a thermal element including chemical contents capable of instantly activating through agitation of the chemical contents inciting a chemical reaction to change a temperature of the thermal element for thermal transfer between the thermal element and the fluid between the IV bag and a patient; and
IV tubing having a hollow core that forms a conduit for a flow of the fluid through the IV tubing from the IV bag to the patient, wherein at least a portion of the IV tubing is coiled around the thermal element to provide a predetermined surface area of engagement between the IV tubing and the thermal element for thermal transfer between the thermal element and the fluid received by the patient when the thermal element is instantly activated and the fluid is flowing through the IV tubing.

18. The system of claim 17 further comprising an insulating wrapper disposed around the thermal element and the portion of the IV tubing coiled around the thermal element.

19. (canceled)

20. A method comprising:
coiling at least a portion of tubing around a thermal element, the tubing having a hollow core that forms a conduit for a flow of fluid through the tubing, the thermal element including chemical contents capable of instantly activating through agitation of the chemical contents inciting a chemical reaction to change a temperature of the thermal element for thermal transfer between the thermal element and fluid in the tubing;
wrapping an insulating wrapper around the portion of the tubing coiled around the thermal element such that a first end of the tubing and a second end of the tubing are unwrapped and exposed to facilitate a connection of the tubing with a fluid flow system; and
providing a first connector on the first end of the tubing and a second connector on the second end of the tubing, the first and second connectors configured for engagement with components of the fluid flow system.

21-25. (canceled)

26. The device of claim 1 wherein agitation of the chemical contents is manually applied to the thermal element by a user.

27. The device of claim 1 wherein agitation of the chemical contents includes one or more of a shaking of the thermal element, a squeezing of the thermal element, a torqueing of the thermal element, a movement of the thermal element, and an application of pressure to the thermal element.

28. The device of claim 1 wherein the thermal element includes one or more of a package, a sack, and a bag containing the chemical contents.

29. The system of claim 17 wherein the fluid is selected from the group consisting of a sodium chloride solution, a dextrose solution, oxygen, and a blood product.

30. The method of claim 20 further comprising activating the thermal element by agitating the chemical contents to change the temperature of the thermal element.
